(19) United States
(12) Patent Application Publication
(10) Pub. No.: US 2009/0247893 A1
(43) Pub. Date: Oct. 1, 2009

(54) METHOD AND APPARATUS FOR MEASURING RESPONSIVENESS OF A SUBJECT

(75) Inventors: Petteri Lapinlampi, Espoo (FI); Mika Sarkela, Espoo (FI)

Correspondence Address: Andrus, Sceales, Starke & Sawall, LLP, 100 East Wisconsin Avenue, Suite 1100, Milwaukee, WI, 53202-4178, US

(73) Assignee: THE GENERAL ELECTRIC COMPANY, Schenectady, NY (US)

(21) Appl. No.: 12/056,928

(22) Filed: Mar. 27, 2008

(51) Int. Cl. A61B 5/0476; A61B 5/0488

(52) U.S. Cl. 600/544; 600/546

(57) ABSTRACT

The invention relates to a method and apparatus for measuring the responsiveness of a subject. Physiological signal data is obtained from a subject through at least one electrode attached to the skin of the subject and a first measure is derived from the physiological signal data, the first measure being indicative of signal energy in the physiological signal data on a frequency band where EMG activity appears. Recorded sequence of the first measure is subjected as input data to a morphological pattern extraction operation. The output data of the operation is indicative of presence of predetermined response waveforms in the input data and a measure of responsiveness is determined based on the output data.

### FIELD OF THE INVENTION

The present invention relates generally to the assessment of the responsiveness of a subject. The invention is typically used in monitoring of subjects with lowered level of consciousness but the invention may also be used for determining the responsiveness of subjects with normal or near normal level of consciousness.

### BACKGROUND OF THE INVENTION

Electroencephalography (EEG) is a well-established method for assessing brain activity. When measurement electrodes are attached to the skin of the skull surface, the weak biopotential signals generated in the pyramid cells of the cortex may be recorded and analyzed. The EEG has been in wide use for decades in basic research of the neural systems of the brain as well as in the clinical diagnosis of various central nervous system diseases and disorders.

Electromyography (EMG) is a method for recording electrical biopotentials of muscles. In an EMG measurement, the electrodes are attached onto the surface of the skin overlying a muscle or directly to the muscle. When a biopotential signal is recorded from the forehead of a subject, the recorded signal indicates both the activity of the facial muscles (FEMG) and the brain (EEG).

One of the special applications of the EEG, which has received attention recently, is the use of a processed EEG signal for objective quantification of the amount and type of brain activity for the purpose of determining the level of consciousness of a patient. In its simplest form, the utilization of an EEG signal allows automatic detection of the alertness of an individual, i.e. if he or she is awake or asleep. This has become an issue of increased interest, both scientifically and commercially, in the context of measuring the depth of hypnosis induced by anesthesia during surgery.

The depth of hypnosis is not directly measurable. Therefore, the level of hypnosis has to be derived from a surrogate signal or from indirectly measured parameters. The most common and popular surrogate signal for this purpose is the EEG, from which several parameters may be determined. The basic reason for the insufficiency of a single parameter is the variety of drugs and the complexity of the drug effects on the EEG signal in human brains. However, during the past few years, some commercial validated devices for measuring the level of consciousness and/or awareness in clinical set-up during anesthesia or sedation have become available. Such devices, which are based on a processed EEG signal and examine the signal as a whole with its multiple features, are marketed by GE Healthcare Finland Oy, Kuortaneenkatu 2, FIN-00510 Helsinki (Entropy®) and by Aspect Medical Systems, Inc., 141 Needham Street, Newton, Mass. 02464, U.S.A. Bispectral Index™ (BIS™) is a trademark of Aspect Medical Systems, Inc.

In addition to the EEG signal data, EMG signal data obtained from facial muscles (fEMG) of the forehead is used for monitoring purposes during anesthesia and intensive care. Recovering facial muscle activity is often the first indicator of the patient approaching consciousness. When this muscle activity is sensed by electrodes placed appropriately, it provides an early indication that the patient is emerging from anesthesia. Similarly, these electrodes can sense pain reactions when the anesthesia is not adequate due to inadequate analgesia. So, the EMG signals give an early warning of the arousal of the patient, and they may also be indicative of inadequate analgesia.

Several factors may affect the state of the central nervous system (CNS) of a patient: sedative drugs, natural sleep cycles, and brain disorders all have their effect on the EEG signal. So far, no methods exist to distinguish these components from each other to provide a clinician an overall picture of the CNS state of the patient. The development of such a method is challenging due to the non-specificity of the EEG signal. A slow wave EEG pattern, for example, may be associated with a high level of a sedative drug, deep natural sleep, or a severe stage of encephalopathy. Correspondingly, low EEG entropy or BIS Index levels may be associated with any of these causes. Furthermore, natural variations of vigilance cause high fluctuations in entropy or BIS Index, which tend to mask any underlying information of the sedative drug effect. Therefore, the above-mentioned devices for measuring the level of (un)consciousness and/or (un)awareness are not suitable for distinguishing the different causes giving rise to the level measured.

The clinician can distinguish between the different causes by including contextual information and by stimulating the patient. For example, if a patient with a slow wave EEG has not received substantial amounts of sedative drugs and has normally functioning liver/kidneys, he cannot be too deeply sedated. If the patient in such a situation anyway does not respond to a strong external stimulus, the clinician may conclude that the patient has developed a brain disorder. To estimate the sedative drug effect and particularly to avoid too deep levels of sedation, it is recommended that some kind of stimulus-response-based scoring is regularly performed by the nursing staff. Such scores are, however, often imprecise and subjective and do not provide continuous information. Furthermore, known stimulus-response-based scoring mechanisms are difficult to implement in automatic monitoring.

A method and apparatus for measuring the responsiveness of a subject is known, in which naturally occurring arousals of the subject are monitored based on changes occurring in a measure indicative of the level of consciousness of the subject. As the method is performed without proactively inducing arousals, it is suitable for automatic detection of responsiveness. The method further improves the specificity of the measurement, since it is known that sedated and naturally sleeping patients react differently to the unintentional stimuli occurring in a clinical environment. However, the specificity of the method to the various underlying physiological response events is compromised, since the measurement is based only on changes in the level of consciousness within a certain time period.

The present invention seeks to alleviate the above drawback and to improve the specificity of the measurement of responsiveness with respect to the actual physiological responses and their types.

### SUMMARY OF THE INVENTION

The present invention seeks to provide a novel mechanism that provides improved specificity to the underlying physiological responses in the estimation of the responsiveness of a subject. The method further seeks to provide a mechanism that allows detection of certain types of responses.

The present invention rests on the discovery that the responses that can be seen in signal energy on a frequency band where EMG activity appears have a typical pattern that shows low inter-subject variability. In the present invention, a measure indicative of signal energy on the said frequency band is first determined. Desired signal waveforms are then extracted from the time series of the measure by subjecting the time series to a morphological pattern extraction operation. A measure of responsiveness is then determined based on the output data of the operation. The “morphological pattern extraction operation” here refers to any operation or process that is capable of detecting desired response waveforms, i.e. capable of providing output data indicative of presence of desired response waveforms in the input data of the operation. The entity carrying out the operation is in this context termed the morphological pattern extractor. As discussed below, the morphological pattern extractor may be implemented as a morphological filter or filter bank, for example, which is typically a wavelet filter or filter bank. Morphological filters are typically used in image processing. Since the morphological filter may be implemented as a filter or filter bank, the term “filter” is in this context intended to cover both alternatives.

Thus one aspect of the invention is providing a method for measuring the responsiveness of a subject. The method comprises obtaining physiological signal data through at least one electrode attached to the subject, deriving a first measure from the physiological signal data, the first measure being indicative of signal energy in the physiological signal data on a frequency band where EMG activity appears, and recording a sequence of the first measure. The method further comprises subjecting the sequence of the first measure to a morphological pattern extraction operation, wherein output data of the morphological pattern extraction operation is indicative of presence of predetermined response waveforms in the sequence of the first measure, and determining a measure of responsiveness based on the output data of the morphological pattern extraction operation.

Another aspect of the invention is that of providing an apparatus for measuring the responsiveness of a subject. The apparatus comprises a data processing unit configured to derive a sequence of a first measure from physiological signal data obtained from a subject through at least one electrode attached to the subject, the first measure being indicative of signal energy in the physiological signal data on a frequency band where EMG activity appears. The apparatus further comprises a morphological pattern extractor configured to provide output data indicative of presence of predetermined response waveforms in input data of the morphological pattern extractor, wherein the morphological pattern extractor is further configured to receive the sequence of the first measure as the input data and a determination unit configured to determine a measure of responsiveness based on the output data of the morphological pattern extractor.

The invention improves the reliability of the measurement of responsiveness, since the physiological responses can be detected more accurately and unequivocally than before from a biopotential signal measured from the subject. Furthermore, the invention enables the detection of desired response patterns from the biopotential signal.

A further advantage relating to the filter-based embodiments of the invention is that the use of a morphological filter or filter bank enables an efficient and uncomplicated artifact removal.

A further aspect of the invention is that of providing a computer program product by which known patient monitoring devices may be upgraded and thus their applicability extended. The program product comprises a first program code portion configured to record a sequence of a first measure indicative of signal energy in physiological signal data on a frequency band where EMG activity appears, wherein the physiological signal data is obtained from a subject through at least one electrode attached to the subject. The program product further comprises a second program code portion configured to provide, based on the sequence of the first measure, output data indicative of presence of predetermined response waveforms in the sequence, and a third program code portion configured to determine a measure of responsiveness based on the output data.

The physiological signal data measured from the subject is typically measured from the forehead of the patient and includes brain wave signal data, such as EEG signal data, and EMG signal data originating from frontal muscles. Responsiveness may also be determined in connection with an ECG measurement, for example, by extracting response waveforms of ECG energy/power on a frequency band where EMG is a dominant signal component. Furthermore, dedicated electrodes may be used for the measurement of responsiveness.

Other features and advantages of the invention will become apparent by reference to the following detailed description and accompanying drawings.

### BRIEF DESCRIPTION OF THE DRAWINGS

In the following, the invention and its preferred embodiments are described more closely with reference to the examples shown in FIG. 1 to 6 in the appended drawings, wherein:

FIG. 1 is a flow diagram illustrating one embodiment of the method of the invention;

FIG. 2 illustrates a typical response pattern measured from the forehead on a frequency band where EMG activity appears;

FIG. 3 illustrates an embodiment of the invention employing discrete wavelet transform;

FIG. 4 illustrates an embodiment of the invention employing stationary wavelet transform;

FIG. 5 illustrates one embodiment of the apparatus/system according to the invention; and

FIG. 6 illustrates the operational entities in the control unit of FIG. 5 for obtaining a measure of responsiveness based on the biopotential signal data measured from the subject.

### DETAILED DESCRIPTION OF THE INVENTION

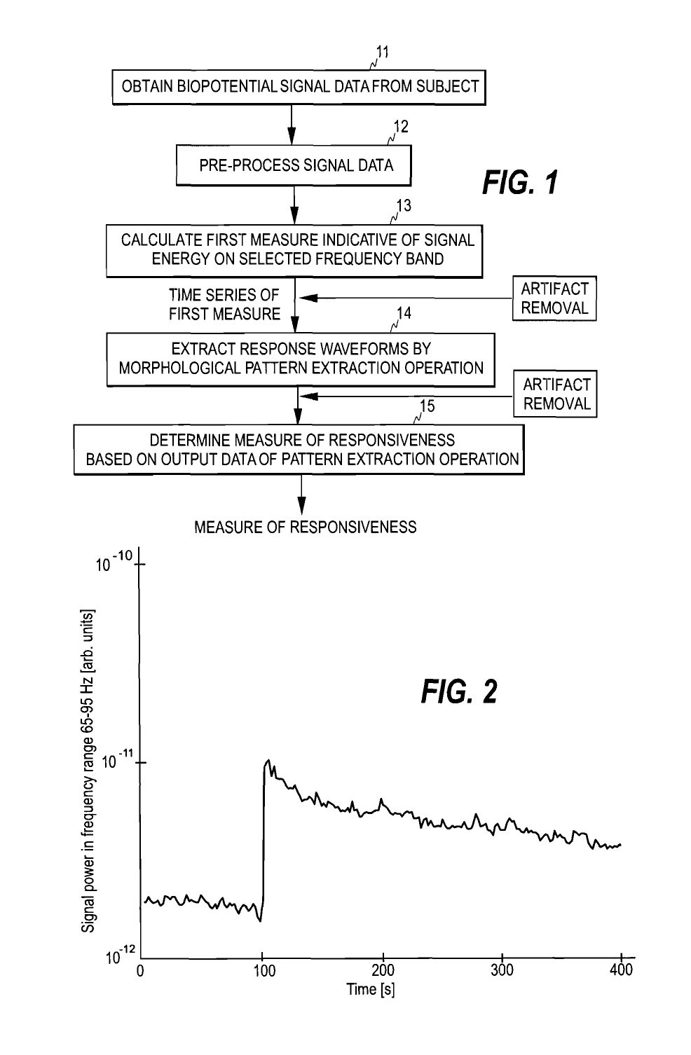

FIG. 1 illustrates one embodiment of the method of the invention. A biopotential signal measured from a subject is first digitized (step 11). The signal is measured through electrodes attached to the subject. Since the electrodes may be either surface electrodes attached to surface of the skin or needle electrodes penetrating the skin surface, the term “electrode” is in this context intended to cover both alternatives. The sampled signal may then be filtered to exclude high- and low-frequency artifacts (step 12). As is common in the art, the digitized signal samples are processed as sets of sequential signal samples representing finite time blocks or time windows, commonly termed “epochs”. It is assumed here that the biopotential signal is measured from the forehead of the subject, i.e. that the signal data contains brain wave signal components and also EMG signal components whenever EMG activity is present.

As mentioned above, the present invention rests on the discovery that the responses that can be seen in signal energy on a frequency band that contains high frequency brain wave signal components and/or EMG signal components have a typical pattern that shows low inter-subject variability. A typical response pattern manifests in the signal power of the said frequency band as a sharp rise followed by a nearly exponential decay. The time constant of the decay and the magnitude of the rise may vary depending on the strength of the stimulus and the state of the patient.

Consequently, the digitized and possibly pre-processed brain wave signal data is first used to calculate a first measure indicative of signal energy or power on a frequency band that that contains high frequency brain wave signal components and/or EMG signal components (step 13). The limits of the frequency band may vary as long as EMG is, whenever present, a major signal component on the band. Since most EMG exists on a frequency band above 10 Hz, the selected frequency band may range from 10 Hz to 200 Hz, for example, more specifically from 50 Hz to 120 Hz, or even more specifically from 65 Hz to 95 Hz. FIG. 2 illustrates an example of a response pattern measured from a subject. In this example, the vertical axis represents the power of the measured brain wave signal data in the frequency range from 65 Hz to 95 Hz and the horizontal axis represents time in seconds. As mentioned above, a typical response pattern comprises a sharp rise followed by a nearly exponential decay, which can clearly be seen in the figure.

The calculation of the first measure may be carried out in various ways. In one embodiment of the invention, the time series of the first measure is obtained by a wavelet filter, as the sum of the squared wavelet coefficients corresponding to a frequency band on which high frequency brain wave signal components and/or EMG signal components appear. The conventional Fast Fourier Transform may also be used at step 13 to produce the first measure. The time series of the first measure may also be calculated straight from the time-domain signal, by utilizing appropriate filters. A time series representing the signal energy on the desired frequency band is thus obtained from step 13.

At step 14, the said time series is then subjected to a morphological pattern extraction operation for extracting response waveforms from the time series. As mentioned above, the morphological pattern extraction operation is typically implemented as a filter or filter bank. The filter or filter bank typically comprises a wavelet filter, but any filter or filter bank that is capable of providing output data indicative of presence of predetermined response waveforms in the input data of the filter may be employed. For example, the filter may be a FIR or IIR filter with desired impulse response. The morphological pattern extraction operation may also be implemented by template matching or principal component analysis, or by using AR (autoregressive) and ARMA (autoregressive moving average) models.

The output data of the morphological pattern extractor is then supplied to a further calculation process (step 15), in which a measure of responsiveness is determined based on the output data. The measure of responsiveness may be calculated as the number of response patterns found by the morphological pattern extractor in a predetermined time window. The strength of the responses may also be incorporated in the calculation. In case of template matching, for example, this may be performed by quantifying the strength of the extracted response patterns as the maximum signal power related to each response.

Artifacts may be removed from the time series of the first measure and/or from the output data of the morphological pattern extractor, as is shown in the figure. The use of wavelet technique for producing the time series of the first measure is advantageous in the sense that removing certain wavelet coefficients may be straightforwardly used to filter artifacts, such as outliers, from the signal to be input to the morphological pattern extraction operation. These outliers include, for example, peaks related to myoclonic twitches that are rather a sign of neurological disorders than true responsiveness of the patient. Possibly remaining artifacts may also be removed by filtering the wavelet coefficients to be supplied to the further calculation process 15. For example, by utilizing the Haar mother wavelet in the morphological filter bank sudden drops in the EMG power caused by a dosage of muscle relaxants can be excluded from the determination of the measure of responsiveness based on the sign of the detail coefficients.

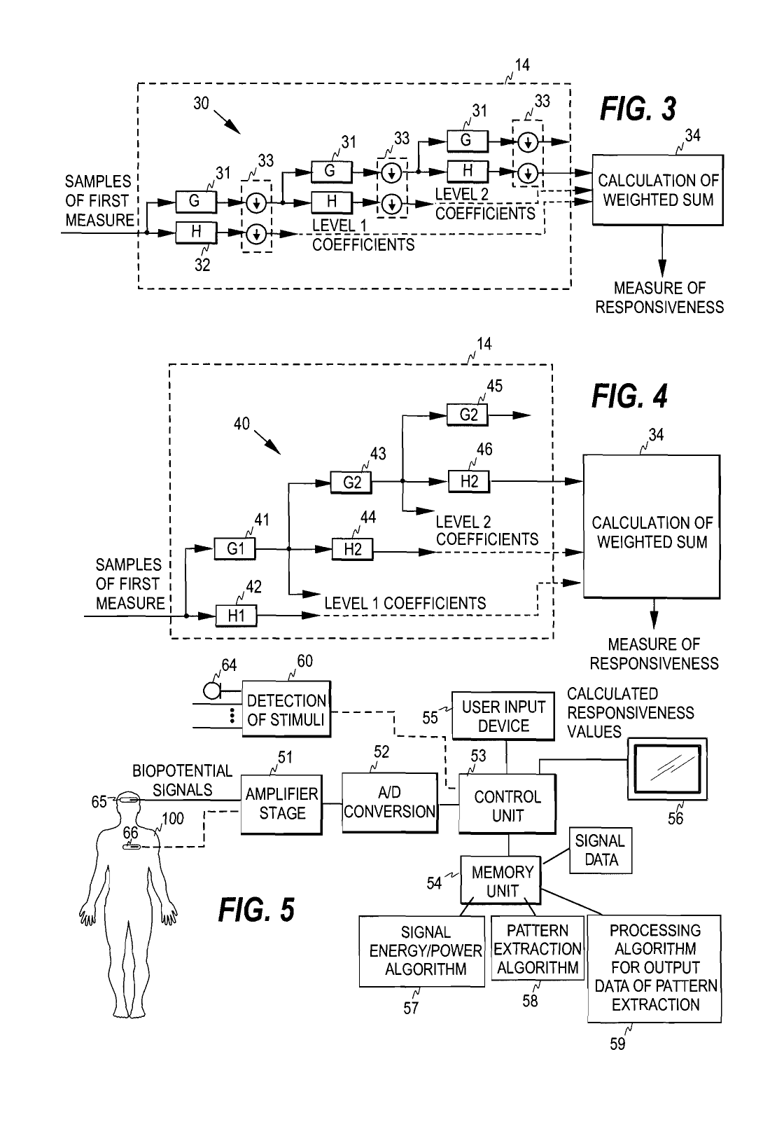

FIG. 3 illustrates an embodiment of the invention employing discrete wavelet transform filter bank 30 according to the Mallat algorithm for carrying out the morphological pattern extraction operation. The process first collects a set of samples of the first measure and supplies the set to the first subband coding process of the filter. Subband coding here refers to the filtering and downsampling operations performed at each decomposition level of a discrete wavelet transform. As common in discrete wavelet transforms, at each decomposition level the signal is first passed through a high-pass filter 32 with impulse response H and a low-pass filter 31 with impulse response G. The two filters are known as a quadrature mirror filter. The impulse responses depend on the mother wavelet used. After the filtering, part of the samples, typically half, is discarded in a downsampling process 33. The downsampled output of the high-pass filter 32 constitutes the wavelet coefficients (so-called detail coefficients) of the respective decomposition level, while the downsampled output of the low-pass filter 31 (so-called approximate coefficients) may be supplied to the next decomposition level. In the embodiment of FIG. 3, the detail coefficients of the third decomposition level are utilized to calculate the measure of responsiveness. However, any combination of the decomposition levels may be used for calculating the said measure and the detail coefficients of different decomposition levels may be weighted differently, as is illustrated in the figure by dashed arrows. Thus, the invention enables a multiresolution analysis that may detect desired waveforms corresponding to different time scales. This enables efficient detection of waveforms of desired types.

In the example of FIG. 3, the detail coefficients of the third decomposition level are supplied to a further processing stage 34 in which the measure of responsiveness is determined based on the sum of the coefficients. The measure may be calculated, for example, as the sum of the coefficients, as the sum of squared or absolute coefficients, as the sum of the logarithms of the coefficients, as a weighted sum of the coefficients, as a weighted sum of the logarithms of the coefficients or a combination of these. In one embodiment of the invention, the weighting of the coefficients or their logarithms is carried out so that the latest coefficients (i.e. the coefficients that correspond to recent samples of the first measure) are weighted more than the coefficients corresponding to less recent samples of the first measure. In one embodiment, the weights are chosen to increase monotonically from the less recent samples to the latest samples. The increase may be linear or nonlinear (e.g. exponential). The weights may also be chosen based on the magnitude of the coefficients. For example, by setting the weights of very small coefficients to zero their contribution to the responsiveness index can be eliminated. Another possibility is to give more weight to small coefficients to enhance the more subtle changes in the first measure. The (weighted) sum obtained may further be scaled to a predetermined scale, such as [0 to 100], thereby to provide more illustrative responsiveness values to the user. Low values then indicate that no or few response patterns were detected.

FIG. 4 illustrates another embodiment of the invention employing a stationary wavelet transform filter bank 40 for extracting the response waveforms. This embodiment corresponds to the embodiment of FIG. 3, except that in the stationary wavelet transform filter bank the signal is not downsampled. Instead, the filters are upsampled (filter length is increased) at each level of decomposition. Thus, at each level of decomposition, the filters are upsampled versions of the filters at the previous decomposition level. In other words, filter 45 is an upsampled version of filter 43 which is an upsampled version of filter 41 and filter 46 is an upsampled version of filter 44 which is an upsampled version of filter 42.

In the embodiments of FIG. 3 and FIG. 4, the number of samples input to the filter bank may be, for example, 256. The original biopotential signal may be sampled at a sampling frequency fs=400 Hz, for example. If the signal energy (i.e. first measure) is determined, for example, in epochs of 5 seconds in step 13, i.e. if one sample to be input to the filter bank is obtained in every 5 seconds, the collection of 256 new samples takes about 21 minutes. However, a new set of 256 samples may be input to the filter bank every 5 seconds, each set including one new sample as compared to the preceding set. In this way, a new set of coefficients (and a new value of responsiveness) is obtained every 5 seconds.

In the embodiment of FIG. 4, the number coefficients obtained from each decomposition level corresponds to the number of input samples, i.e. in this example 256 samples are obtained for the further processing stage 34, each coefficient corresponding to the respective input sample. In the embodiment of FIG. 3, the number of coefficients is reduced by half when proceeding to the next level. Thus, the number of coefficients obtained from the third decomposition level is 32, i.e. each coefficient corresponds to 8 input samples.

As discussed above, the wavelet transform may be fine-tuned for the detection of desired signal morphologies in the time window of interest by proper selection of the mother wavelet and the scale(s), and possibly also by weighting the different decomposition levels differently. When the biopotential signal is measured from the forehead, the mother wavelet to be used for the wavelet transform belongs preferably to the Daubechies (db) family, since this family includes wavelets that have a good match for the actual response waveform in the time series of the first measure. In the above examples, in which the length of the time window is 5 seconds, use of Daubechies 2 or Daubechies 1 mother wavelet and decomposition level 3 proved to be able to detect relevant responses in the time series of the first measure, and to ignore very short events in the said time series.

FIG. 5 illustrates one embodiment of the system or apparatus according to the invention. The biopotential signal(s) obtained from one or more sensors attached to a patient 100 are supplied to an amplifier stage 51, which amplifies the signal(s) before they are sampled and converted into digitized format in an A/D converter 52. The digitized signals are supplied to a control unit 53 which may comprise one or more processor (or microprocessor) based systems. As noted above, the signal data is typically measured through electrodes attached to the forehead of the patient, i.e. the signal data contains typically brain wave signal data, such as EEG data, and EMG signal components resulting from the activity of the facial muscles. Instead of EEG data magnetoencephalographic (MEG) signal data may also be measured. MEG is indicative of the magnetic component of brain activity, i.e. it is the magnetic counterpart of EEG.

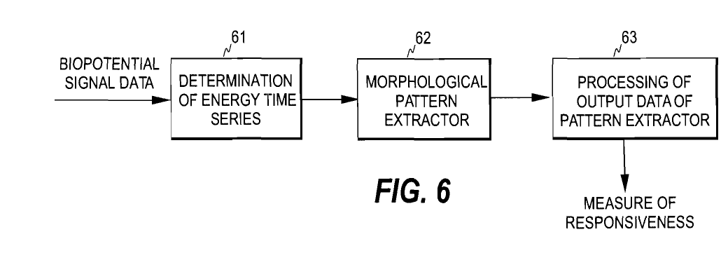

The control unit is provided with a memory or database 54 holding the digitized signal data obtained from the electrodes. The memory or database may also store a first algorithm 57 for calculating the power/energy time series, a second algorithm 58 for implementing the morphological pattern extraction operation, and a third algorithm 59 for processing the output data of the morphological pattern extraction operation. The resulting measure/index of responsiveness may be displayed on the screen of a monitor 56. Various parameters possibly needed in the calculation of the responsiveness may also be supplied through an input device 55, such as a keyboard, if the computer unit has no access to such data. In terms of the invention, the control unit, equipped with the above algorithms, may thus be seen as an entity of three consecutive operational modules or units, as is shown in FIG. 6: a data processing unit 61 configured to determine the power/energy time series of the desired EEG/EMG frequency band, a morphological pattern extractor 62 configured to detect presence of desired response waveforms in the time series, and a determination unit 63 configured to determine the measure of responsiveness based on the output signal of the morphological pattern extractor.

Although one computer-based apparatus may perform the above steps, the processing of the data may also be distributed among different units/processors (servers) within a network, such as a hospital LAN (local area network). The apparatus of the invention may thus also be implemented as a distributed system.

The above determination of the measure of responsiveness may be used regardless of whether arousals are produced in the subject intentionally or unintentionally. However, as the method is suitable for automatically and continuously monitoring the responsiveness and as it does not interfere with clinical procedures, it is advantageous that the arousals are caused without proactive actions of the clinical staff. The measure of responsiveness may be determined continuously or when a stimulus (such as noise) is detected in the clinical environment. If the determination of the responsiveness is not continuous but synchronized with the occurrence of the stimuli, the apparatus/system is further provided with a detection system 60 for detecting the stimuli that may cause the arousals in the clinical environment. The detection system may detect one or more stimulus types, in which case it is provided with one or more detection interfaces for receiving signals indicative of the stimuli. For example, if audio stimuli occurring in the clinical environment are detected, the corresponding detection interface may be provided with a microphone 64 for measuring the noise level. Some of the detection interfaces may be connected to desired clinical equipment, such as to a ventilator for measuring the airway pressure for detecting patient/ventilator dyssynchrony or to a blood pressure cuff for detecting when the cuff is pressurized.

A conventional patient monitor may also be upgraded to enable the monitor to determine the measure of responsiveness. Such an upgrade may be implemented by delivering to the patient monitor a plug-in software module that determines a responsiveness measure in the above-described manner based on the biopotential signal data stored in the device. The software module, which typically comprises the algorithms 57-59 in the form of program code, may be delivered, for example, on a data carrier, such as a CD or a memory card, or through a telecommunications network.

A responsiveness monitor of the invention may also be implemented as a separate module that may be connected to a conventional patient monitor so as to retrieve the biosignal data measured by the patient monitor. The responsiveness monitor may comprise a display of its own for displaying the calculated responsiveness measure to the user, and it may optionally include the above-described detection system 60 for determining the responsiveness non-continuously upon detection of a stimulus.

Although the determination of responsiveness is typically carried out in connection with measurement of brain wave signal data, the method of the invention may also be carried out in connection with ECG measurement, for example, by extracting response waveforms of ECG power on a frequency band where EMG is a dominant signal component. Furthermore, it is not necessary that the electrodes are simultaneously used to collect other physiological data, but dedicated electrodes attached on top of a desired muscle may be used for determining responsiveness according to the method of the invention. The time series of a measure indicative of signal energy on a frequency band where EMG activity appears may thus be obtained through EEG electrodes or MEG sensors 65, ECG electrodes 66, or through dedicated responsiveness electrodes that may be attached on the forehead, for example. The number and position of the electrodes may vary according to known measurement arrangements.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural or operational elements that do not differ from the literal language of the claims, or if they have structural or operational elements with insubstantial differences from the literal language of the claims.

1. A method for measuring the responsiveness of a subject, the method comprising:
obtaining physiological signal data through at least one electrode attached to the subject;
deriving a first measure from the physiological signal data, the first measure being indicative of signal energy in the physiological signal data on a frequency band where EMG activity appears;
recording a sequence of the first measure;
subjecting the sequence of the first measure to a morphological pattern extraction operation, wherein output data of the morphological pattern extraction operation is indicative of presence of predetermined response waveforms in the sequence of the first measure; and
determining a measure of responsiveness based on the output data of the morphological pattern extraction operation.

2. A method according to claim 1, wherein the obtaining includes obtaining the physiological signal data, in which the physiological signal data includes high frequency EEG signal data.

3. A method according to claim 1, wherein the deriving includes deriving the first measure, in which the first measure represents power of the physiological signal data on said frequency band.

4. A method according to claim 1, wherein the subjecting includes subjecting the sequence of the first measure to the morphological pattern extraction operation, wherein the morphological pattern extraction operation is implemented as a filter.

5. A method according to claim 4, wherein the subjecting includes subjecting the sequence of the first measure to the filter, wherein the filter is configured to perform a wavelet transform.

6. A method according to claim 5, wherein the determining includes selecting output coefficients of the filter from at least one decomposition level of the filter.

7. A method according to claim 6, wherein the determining comprises determining the measure of responsiveness based on a sum of the selected output coefficients.

8. A method according to claim 7, wherein the determining comprises weighting the selected output coefficients.

9. A method according to claim 8, wherein the weighting comprises giving more weight to output coefficients that correspond to most recent values in the sequence of the first measure, wherein the output coefficients belong to the selected output coefficients.

10. An apparatus comprising:
a data processing unit configured to derive a sequence of a first measure from physiological signal data obtained from a subject through at least one electrode attached to the subject, the first measure being indicative of signal energy in the physiological signal data on a frequency band where EMG activity appears;
a morphological pattern extractor configured to provide output data indicative of presence of predetermined response waveforms in input data of the morphological pattern extractor, wherein the morphological pattern extractor is further configured to receive the sequence of the first measure as the input data; and
a determination unit configured to determine a measure of responsiveness based on the output data of the morphological pattern extractor.

11. An apparatus according to claim 10, wherein the physiological signal data includes high frequency EEG signal data.

12. An apparatus according to claim 10, wherein the first measure represents power of the physiological signal data on said frequency band.

13. An apparatus according to claim 10, wherein the morphological pattern extractor comprises a filter.

14. An apparatus according to claim 13, wherein the filter is configured to perform a wavelet transform.

15. An apparatus according to claim 14, wherein the determination unit is configured to select output coefficients of the filter from at least one decomposition level of the filter.

16. An apparatus according to claim 15, wherein determination unit is configured to determine the measure of responsiveness based on a sum of the selected output coefficients of the filter.

17. An apparatus according to claim 16, wherein the determination unit is configured to determine the measure of responsiveness based on a weighted sum of the selected output coefficients.

18. An apparatus according to claim 17, wherein the determination unit is configured to put more weight on output coefficients that correspond to most recent values of the first measure, wherein the output coefficients belong to the selected output coefficients.

19. A computer program product for a patient monitor, the computer program product comprising:
a first program code portion configured to record a sequence of a first measure indicative of signal energy in physiological signal data on a frequency band where EMG activity appears, wherein the physiological signal data is obtained from a subject through at least one electrode attached to the subject;
a second program code portion configured to provide, based on the sequence of the first measure, output data indicative of presence of predetermined response waveforms in the sequence; and
a third program code portion configured to determine a measure of responsiveness based on the output data.
